SEQUENCE LISTING (45 sequences)

SEQ ID NO: 1 - protein, 23 aa, Artificial Sequence
  Synthetic polypeptide
GALFLGWLGAAGSTMGAKKKRKV

SEQ ID NO: 2 - protein, 16 aa, Artificial Sequence
  Synthetic polypeptide
RQIKIWFQNRRMKWKK

SEQ ID NO: 3 - protein, 7 aa, Artificial Sequence
  Synthetic polypeptide
RRMKWKK

SEQ ID NO: 4 - protein, 16 aa, Artificial Sequence
  Synthetic polypeptide
RRWRRWWRRWWRRWRR

SEQ ID NO: 5 - protein, 18 aa, Artificial Sequence
  Synthetic polypeptide
RGGRLSYSRRRFSTSTGR

SEQ ID NO: 6 - protein, 11 aa, Artificial Sequence
  Synthetic polypeptide
YGRKKRRQRRR

SEQ ID NO: 7 - protein, 9 aa, Artificial Sequence
  Synthetic polypeptide
RKKRRQRRR

SEQ ID NO: 8 - protein, 11 aa, Artificial Sequence
  Synthetic polypeptide
YARAAARQARA

SEQ ID NO: 9 - protein, 8 aa, Artificial Sequence
  Synthetic polypeptide
RRRRRRRR

SEQ ID NO: 10 - protein, 8 aa, Artificial Sequence
  Synthetic polypeptide
KKKKKKKK

SEQ ID NO: 11 - protein, 27 aa, Artificial Sequence
  Synthetic polypeptide
  misc_feature (25)..(25): Xaa can be any naturally occurring amino acid
GWTLNSAGYLLGKINLKALAALAKXIL

SEQ ID NO: 12 - protein, 18 aa, Artificial Sequence
  Synthetic polypeptide
LLILLRRRIRKQANAHSK

SEQ ID NO: 13 - protein, 16 aa, Artificial Sequence
  Synthetic polypeptide
SRRHHCRSKAKRSRHH

SEQ ID NO: 14 - protein, 11 aa, Artificial Sequence
  Synthetic polypeptide
NRARRNRRRVR

SEQ ID NO: 15 - protein, 15 aa, Artificial Sequence
  Synthetic polypeptide
RQLRIAGRRLRGRSR

SEQ ID NO: 16 - protein, 13 aa, Artificial Sequence
  Synthetic polypeptide
KLIKGRTPIKFGK

SEQ ID NO: 17 - protein, 10 aa, Artificial Sequence
  Synthetic polypeptide
RRIPNRRPRR

SEQ ID NO: 18 - protein, 18 aa, Artificial Sequence
  Synthetic polypeptide
KLALKLALKALKAALKLA

SEQ ID NO: 19 - protein, 14 aa, Artificial Sequence
  Synthetic polypeptide
KLAKLAKKLAKLAK

SEQ ID NO: 20 - protein, 27 aa, Artificial Sequence
  Synthetic polypeptide
GALFLGFLGAAGSTNGAWSQPKKKRKV

SEQ ID NO: 21 - protein, 21 aa, Artificial Sequence
  Synthetic polypeptide
KETWWETWWTEWSQPKKKRKV

SEQ ID NO: 22 - protein, 20 aa, Artificial Sequence
  Synthetic polypeptide
LKKLLKKLLKKLLKKLLKKL

SEQ ID NO: 23 - protein, 34 aa, Artificial Sequence
  Synthetic polypeptide
QAATATRGRSAASRPTERPRAPARSASRPRRPVE

SEQ ID NO: 24 - protein, 23 aa, Artificial Sequence
  Synthetic polypeptide
MGLGLHLLVLAAALQGAKSKRKV

SEQ ID NO: 25 - protein, 26 aa, Artificial Sequence
  Synthetic polypeptide
AAVALLPAVLLALLAPAAANYKKPKL

SEQ ID NO: 26 - protein, 28 aa, Artificial Sequence
  Synthetic polypeptide
MANLGYWLLALFVTMWTDVGLCKKRPKP

SEQ ID NO: 27 - protein, 24 aa, Artificial Sequence
  Synthetic polypeptide
LGTYTQDFNKFHTFPQTAIGVGAP

SEQ ID NO: 28 - protein, 26 aa, Artificial Sequence
  Synthetic polypeptide
  misc_feature (24)..(24): Xaa can be any naturally occurring amino acid
DPKGDPKGVTVTVTVTVTGKGDPXPD

SEQ ID NO: 29 - protein, 14 aa, Artificial Sequence
  Synthetic polypeptide
PPPPPPPPPPPPPP

SEQ ID NO: 30 - protein, 18 aa, Artificial Sequence
  Synthetic polypeptide
VRLPPPVRLPPPVRLPPP

SEQ ID NO: 31 - protein, 10 aa, Artificial Sequence
  Synthetic polypeptide
PRPLPPPRPG

SEQ ID NO: 32 - protein, 30 aa, Artificial Sequence
  Synthetic polypeptide
SVRRRPRPPYLPRPRPPPFFPPRLPPRIPP

SEQ ID NO: 33 - protein, 21 aa, Artificial Sequence
  Synthetic polypeptide
TRSSRAGLQFPVGRVHRLLRK

SEQ ID NO: 34 - protein, 23 aa, Artificial Sequence
  Synthetic polypeptide
GIGKFLHSAKKFGKAFVGEIMNS

SEQ ID NO: 35 - protein, 37 aa, Artificial Sequence
  Synthetic polypeptide
KWKLFKKIEKVGQNIRDGIIKAGPAVAVVGQATQIAK

SEQ ID NO: 36 - protein, 28 aa, Artificial Sequence
  Synthetic polypeptide
ALWMTLLKKVLKAAAKAALNAVLVGANA

SEQ ID NO: 37 - protein, 26 aa, Artificial Sequence
  Synthetic polypeptide
GIGAVLKVLTTGLPALISWIKRKRQQ

SEQ ID NO: 38 - protein, 14 aa, Artificial Sequence
  Synthetic polypeptide
INLKALAALAKKIL

SEQ ID NO: 39 - protein, 33 aa, Artificial Sequence
  Synthetic polypeptide
GFFALIPKIISSPLPKTLLSAVGSALGGSGGQE

SEQ ID NO: 40 - protein, 15 aa, Artificial Sequence
  Synthetic polypeptide
LAKWALKQGFAKLKS

SEQ ID NO: 41 - protein, 27 aa, Artificial Sequence
  Synthetic polypeptide
  misc_feature (23)..(23): Xaa can be any naturally occurring amino acid
SMAQDIISTIGDLVKWIIQTVNXFTKK

SEQ ID NO: 42 - protein, 41 aa, Artificial Sequence
  Synthetic polypeptide
LLGDFFRKSKEKIGKEFKRIVQRIKQRIKDFLANLVPRTES

SEQ ID NO: 43 - protein, 18 aa, Artificial Sequence
  Synthetic polypeptide
PAWRKAFRWAWRMLKKAA

SEQ ID NO: 44 - protein, 18 aa, Artificial Sequence
  Synthetic polypeptide
KLKLKLKLKLKLKLKLKL

SEQ ID NO: 45 - protein, 99 aa, Artificial Sequence
  Synthetic polypeptide
MAGKAGEGEIPAPLAGTVSKILVKEGDTVKAGQTVLVLEAMKMETEINAPTDGKVEKVLVKERDAVQGGQGLIKIGVENLYFQSTMEQKLISEEDLEFT

(19) United States
(12) Patent Application Publication
(10) Pub. No.: US 2021/0238564 A1
(43) Pub. Date: Aug. 5, 2021

(54) TELOMERASE HOLOENZYME COMPLEX AND METHODS OF USE THEREOF

(71) Applicant: The Board of Regents of the University of Texas System, Austin, TX (US)

(72) Inventors: Jerry W. SHAY, Dallas, TX (US); Enzo TEDONE, Dallas, TX (US); Mohammed E. SAYED, Canton, MI (US)

(73) Assignee: The Board of Regents of the University of Texas System, Austin, TX (US)

(21) Appl. No.: 17/273,880

(22) PCT Filed: Sep. 3, 2019
(86) PCT No.: PCT/US19/49271

§ 371 Date: Mar. 5, 2021

Related U.S. Application Data
(60) Provisional application No. 62/727,743, filed on Sep. 6, 2018.

(51) Int. Cl. C12N 9/12; G01N 33/50

(52) U.S. Cl. CPC C12N 9/1276; C12Y 207/07049; G01N 2333/9128; G01N 33/5023; G01N 33/5047

(57) ABSTRACT

The present disclosure describes purified telomerase holoenzyme and its delivery to cells, such as T cells, for increasing telomere length, increasing cell proliferation, and impeding cell senescence.

### PRIORITY CLAIM

This application claims benefit of priority to U.S. Provisional Application Ser. No. 62/727,743, filed Sep. 6, 2018, the entire contents of which are hereby incorporated by reference.

### BACKGROUND

### 1. Field

The present disclosure relates to the fields of cell biology, molecular biology, protein biology and medicine. More specifically, it describes the production and delivery of a telomerase holoenzyme complex to cells to slow or correct telomere shortening.

### 2. Description of Related Art

Telomeres are tandem repeats that cap the end of linear chromosomes to protect them from degradation and to prevent chromosome fusion [1]. In normal human proliferating cells telomeres get progressively shorter with each cell division [2], leading eventually to DNA damage responses, replicative senescence or apoptosis [3]. One consequence of proliferation is that telomere length declines with age [4] and is considered a biomarker of biological (not chronological) age [5] that also correlate with various age-related pathologies including cancer [3], dementia [6, 7] and cardiovascular diseases [5]. Recent studies in mice have shown that by preventing telomere shortening, a single hallmark of aging, both healthspan and lifespan resulted to be increased [8, 9].

Telomerase, the reverse transcriptase involved in de novo addition of telomeric TTAGGG repeats at the end of telomeres, is a ribonucleoprotein enzyme complex that is comprised of two main components, the catalytic protein subunit (TERT), and the template RNA (TR or TERC). In humans, TERT is exclusively expressed in cells that are normally capable of long-term proliferation (e.g., proliferating non quiescent stem cells), but not in normal differentiated somatic cells, except for activated lymphocytes [10, 11].

T lymphocytes (T-cells) are a core cell type in the immune system that mostly circulate in a quiescent non-proliferating state but rapidly divide when activated with antigens or nonspecific stimuli [11]. In vitro, T-cells can be activated and proliferate in response to a specific antigen or to a non-specific (mitogenic anti-CD3 & anti-CD28 antibodies) stimulation [11]. Telomerase activity is transiently upregulated in activated human T-cells but this telomerase is not sufficient to counterbalance telomere loss during rapid cell expansion ultimately leading to replicative senescence both in vitro and in vivo [11, 12]. As such, telomere length and the capability to reactivate telomerase activity are key factors that determine the lifespan of T-cells and antitumor activity of tumor-infiltrating lymphocytes (TILs), which mediate the regression of tumors in patients with healthy immune responses [13, 14]. In fact, TILs with longer telomeres are able to persist in vivo longer and mediate more robust antitumor effects [15].

Given their antitumor abilities, human antigen-specific T-cells are finding increased use as a prime tool for adoptive immunotherapy to treat various forms of cancer and infectious diseases such as AIDS [16, 17]. It is now possible to modify patient's autologous T-cells with cancer antigen-specific T-cell receptor genes, followed by the adoptive transfer of the modified and in vitro expanded T-cells back to the host. However, upon prolonged periods of culturing and expansion in vitro, the modified T cells have a limited replicative potential in vivo and ultimately enter a senescent state (T cell exhaustion), which results from progressive loss of telomere DNA. Since senescing cells have rather limited potential for use in immunotherapy, a technology providing the means to efficiently protect T-cells from telomere loss during the rapid expansion in vitro would be highly advantageous for successful clinical application of antigen-specific T-cells as well as many other types of cells.

### SUMMARY

As described below, the inventors have successfully engineered a biotin-tagged recombinant hTERT and overexpressed it along with hTR (the functional RNA component of telomerase) in the human cell line H1299. They have also developed a 3-step purification procedure strategy to purify the recombinant telomerase from cell lysates. This multi-step purification procedure allowed the inventors to obtain highly enriched, catalytically active enzyme. Importantly, the inventors employed biotin-tag they developed that allowed pulling down not only telomerase (hTERT+hTR) but the whole reconstituted telomerase holoenzyme complex containing other essential telomerase-associated proteins such as dyskerin (DKC1), the ribonucleoprotein NOP10 and NHP2. By using a combination of cell-penetrating peptides and an active uptake mechanism induced by NaCl-mediated hyperosmolarity, the inventors delivered the purified telomerase holoenzyme to normal young and aged human cells (e.g., antigen-stimulated peripheral blood mononuclear cells and lung fibroblasts). Delivered telomerase retained strong activity both in the cytoplasm and nuclear compartment. The inventors also demonstrated that three consecutive deliveries (every three days) of telomerase in vitro were sufficient to significantly extend both telomere length and the cellular replicative lifespan. Importantly, the treatment did not immortalize or transform the cells which ultimately underwent senescence and the delivered telomerase holoenzyme stayed active for a limited time window (up to 24-36 hours). This human recombinant telomerase holoenzyme can be employed to transiently lengthen telomeres and therefore extend the replicative lifespan of aged human cells.

Thus, in accordance with the present disclosure, there is provided a method of increasing telomere length and/or increasing the proliferative capacity of a cell comprising (i) providing a population of cells; (ii) contacting at least a first portion said population of cells with a purified recombinant telomerase holoenzyme; and (iii) measuring the expression of one or more target genes regulated by telomere length in a cell from said first portion. The method may further comprise (iv) introducing a second cell from said first portion into a subject when one or more of said target genes shows an expression profile indicative of telomerase activity as compared to an untreated cell, such as an untreated cell from a second portion of said population of cells.

The method may further comprise measuring the expression of one or more target genes regulated by telomere length in a third cell of said population of cells prior to step (ii). The one or more target genes may be ISG15, TEAD4, PD-1, and/or BAX. The population of cells may be PBMCs. The population of cells may be T cells, such as a CD3⁺/CD28⁺ T cell. The method may further comprise removing said population of cells from a subject prior to step (i). The subject may be a human subject or a humanized mouse, such as a NOD SCID gamma mouse with umbilical cord blood stem cells. The telomerase holoenzyme may be coupled to a cell permeability peptide.

In another embodiment, there is provided a method of increasing a cell's proliferative capacity comprising (i) providing a population of cells; (ii) contacting said a first portion of said population of cells with a recombinant telomerase holoenzyme; (iii) measuring the total number of cell divisions that a first cell from said first portion performs before senescence or apoptosis are triggered; (iv) measuring the total number of cell divisions that a cell from a second but non-telomerase treated portion of said population of cells performs before senescence or apoptosis are triggered; and (v) determining whether a second cell from said first portion does not exhibit a characteristic of cancer. The method may further comprise introducing a third cell from said first portion into a subject when the total number of cell divisions measured in step (iii) is greater than in step (iv), and when said second cell from said first portion does not exhibit a characteristic of cancer.

The method may further comprise measuring telomere length and/or the expression of one or more target genes regulated by telomere length (a) as part of step (iii) or (b) if a fourth cell from said population of cells prior to step (ii). The one or more target genes may be ISG15, TEAD4, PD-1, and/or BAX. The population of cells may be PBMCs. The population of cells may be T cells, such as a CD3⁺/CD28⁺ T cell. The method may further comprise removing said population of cells from a subject prior to step (i). The subject may be a human subject or a humanized mouse, such as a NOD SCID gamma mouse with umbilical cord blood stem cells. The telomerase holoenzyme may be coupled to a cell permeability peptide.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-B. (FIG. 1A) Telomerase activity measured by ddTRAP on stimulated T-cells after stimulation with anti-CD3/anti-CD28 Dynabeads. (FIG. 1B) Telomere length measurements by TeSLA (Telomere Shortest Length Assay) in stimulated T-cells over a 10-day period.

FIG. 2. Correlation between telomerase activity at day 3 after stimulation (peak) and the maximal cell number (a proxy for the rate of cell division) over a 10-day period in peripheral blood mononuclear cells (PBMC) from 114 volunteers 28-113 years-old (unpublished data).

FIGS. 3A-B. (FIG. 3A) Human TERT gene (hTERT). (FIG. 3B) Recombinant hTERT carrying a biotin-tag in the N-terminal domain.

FIG. 4. Purification of human recombinant telomerase holoenzyme.

FIGS. 5A-C. (FIG. 5A) In vitro activity of purified recombinant telomerase holoenzyme measured by ddTRAP. (FIG. 5B) Identification in the major purified complex of both TERT and other telomerase-associated proteins by Western Blot. (FIG. 5C) Individual gels showing components of the telomerase-associated proteins by Western Blot (Dyskerin=DKC1)

FIGS. 6A-D. (FIG. 6A) PBMC composition. (FIG. 6B) In vitro stimulation of T-cells with anti-CD3/anti-CD28 Dynabeads mimics in vivo physiologic stimulation by Antigen Presenting Cells (APC). (FIG. 6C) Unstimulated PBMC show little or no proliferation activity in vitro. (FIG. 6D) Stimulated PBMC with anti-CD3/anti-CD28 Dynabeads rapidly divide in vitro.

FIGS. 7A-C. (FIG. 7A) Gel-based TRAP on stimulated PBMC from a young donor over a 10-day period. (FIG. 7B) ddTRAP on stimulated PBMC from the same donor of figure a. Telomerase activity decrease after day 3 is more easily detected compared to gel-based TRAP. (FIG. 7C) Work-flow of Droplet Digital PCR.

FIGS. 8A-B. (FIG. 8A) Telomere length measured by TRF indicates no telomere length changes in stimulated PBMC over a 10-day period. (FIG. 8B) Telomere length measured by TeSLA (Telomere Shortest Length Assay) indicates progressive telomere shortening in stimulated PBMC over a 10-day period.

FIG. 9. Telomerase activity with or without treatment with telomerase holoenzyme. Control cells (column 1, 3, and 5) have been equally treated with cell penetrating peptides (not conjugated with telomerase) and customized media.

FIG. 10. Telomerase activity from the cytoplasmic and the nuclear fraction of stimulated PBMC with or without treatment with telomerase holoenzyme. Control cells have been equally treated with cell penetrating peptides (not conjugated with telomerase) and customized culture media. *p<0.05 vs untreated

FIG. 11. Average telomere length (Avg) and length of the shortest 20% telomeres (Short. 20%) measured by TeSLA in stimulated PBMC from young healthy adults after three consecutive deliveries of telomerase.

FIG. 12. Average telomere length (Avg) and length of the shortest 20% telomeres (Short. 20%) measured by TeSLA in stimulated PBMC from older healthy individuals after three consecutive deliveries of telomerase.

FIGS. 13A-B. (FIG. 13A) Growth curve of stimulated PBMC from four young adult volunteers treated with telomerase holoenzyme for three consecutive times at days 3, 6, and 9. Average Population Doublings in the Young (mean age 32±2; n=4): 15.9±3.1 PD (Ctrl) vs 22.0±3.0 PD (+telomerase). (FIG. 13B) Growth curve of stimulated PBMC from two older volunteers treated with telomerase holoenzyme for three consecutive times at days 3, 6, and 9. Average Population Doublings in the Old (mean age 65±3; n=2): 10.1±0.5 PD (Ctrl) vs 16.0±1.6 PD (+telomerase).

FIG. 14. Growth curve of aged human IMR-90 treated with telomerase holoenzyme every 3 days.

FIG. 15. Expression level of genes reported to be regulated by telomere length in stimulated PBMC treated with telomerase holoenzyme. *p<0.05

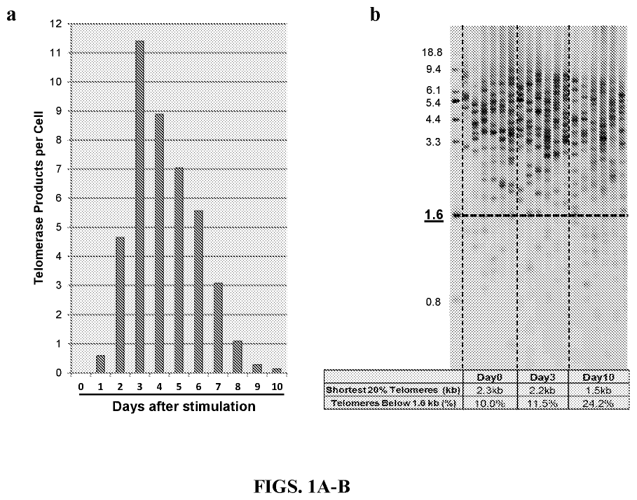

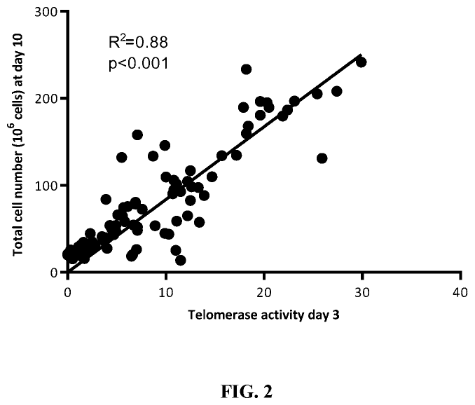

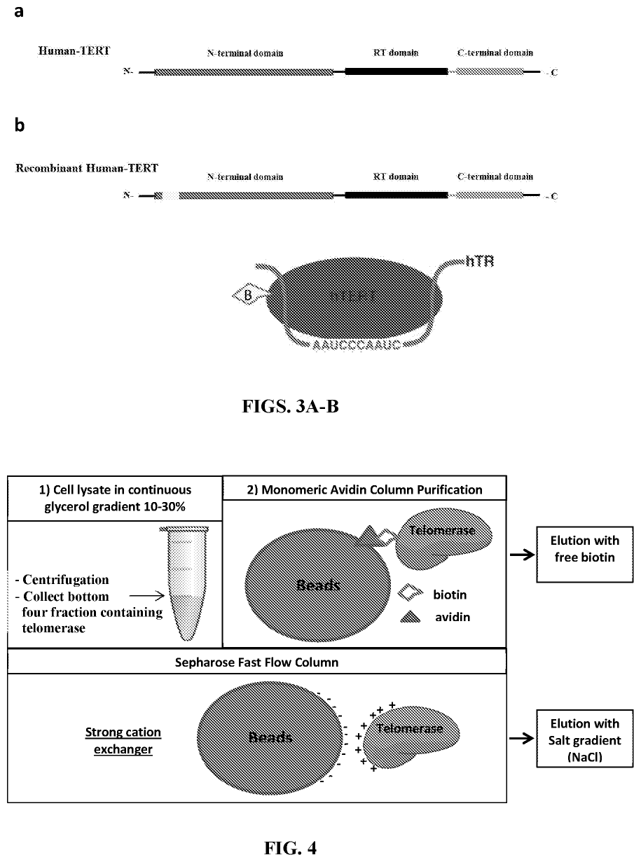

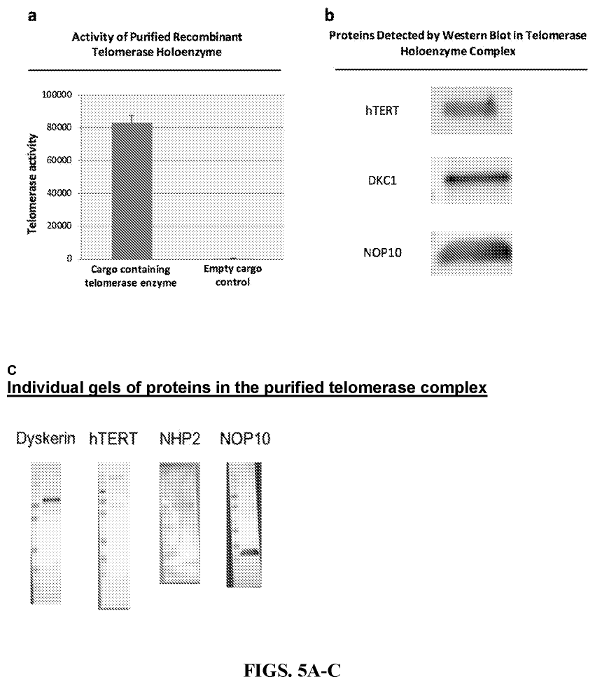

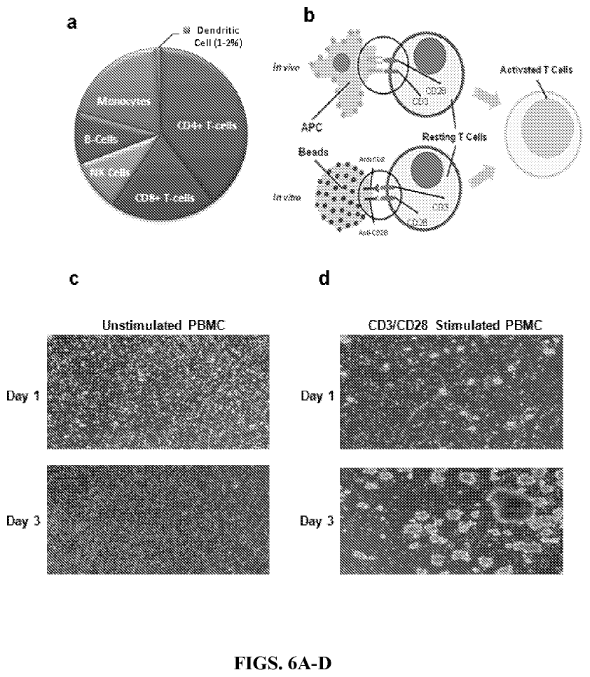

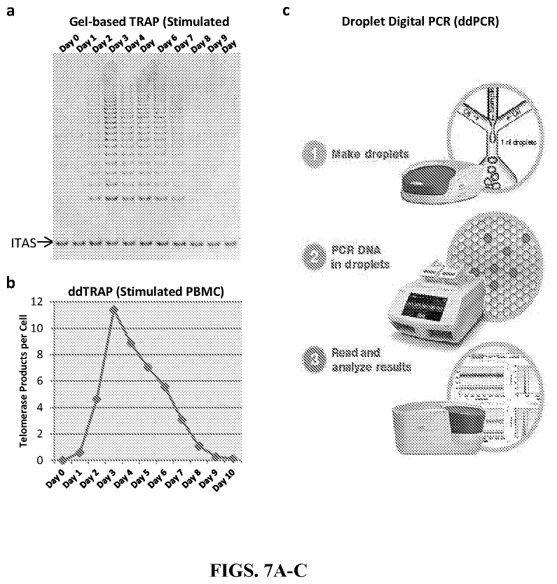

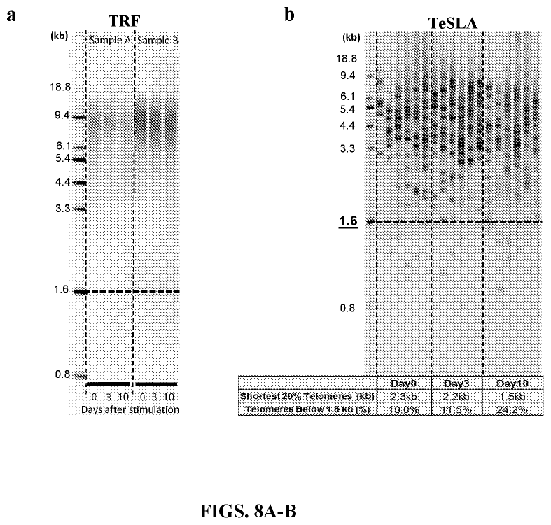

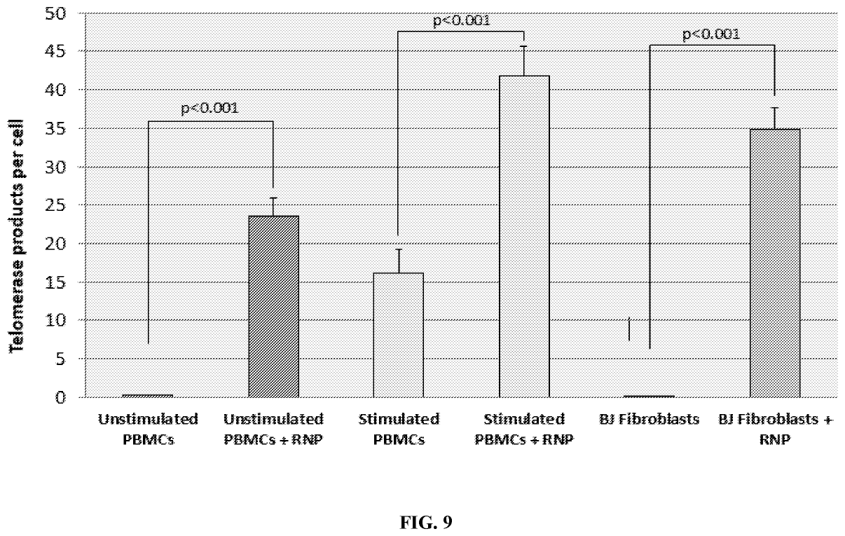

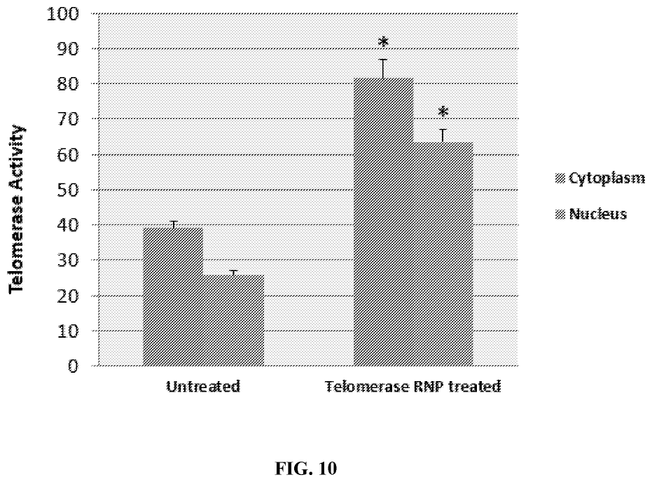

### DETAILED DESCRIPTION OF ILLUSTRATIVE EMBODIMENTS

As discussed above, senescing cells have rather limited potential for use in therapy, such as adoptive immunotherapy. Thus, a technology providing the means to efficiently protect cells from telomere loss during the rapid expansion in vitro would be highly advantageous for successful clinical application of cells like antigen-specific T-cells.

One current strategy, known as ectopic TERT expression by retroviral cell infection (random integration site), has been shown to significantly extend the replicative lifespan of primary human cells [18, 19]. However, many limitations prevent the successful use of retroviral vectors in vivo including their inability to transduce non dividing cells, immunogenic problems and the high risk of insertional mutagenesis, which can cause oncogene activation or tumor-suppressor gene inactivation [20, 21]. Furthermore, strategies for constitutive telomerase reactivation have raised safety concerns due to the close correlation of most cancers and steady expression of endogenous telomerase [22].

Some pharmacological agents such as sex hormones (e.g., testosterone and β-estradiol) and cycloastragenol (extracted from the Chinese root Astragalus) have been reported to slightly upregulate telomerase activity in some, but not all, human cells [23-25]. However, studies performed in stimulated PBMC/T-cells have failed to demonstrate in vitro that the upregulation of telomerase activity induced by any drug promoted, in turn, telomere elongation/maintenance. In addition, potential off-target effects of compounds that activate TERT at a transcriptional level (e.g., through activation of mitogenic pathways that lead to the activation of the oncogene c-myc) may drive cancer [25, 26].

Thus, even though there exist limited preliminary longitudinal studies in human volunteers reporting that the oral administration of sex hormones or cycloastragenol promoted telomere maintenance in peripheral immune cells [27, 28], it is still not clear if telomere length changes were exclusive of immune cells only and why the treatment is successful in some cases but not in others (also manifesting side effects) [29]. Finally, other independent studies have found opposite results and reported that mature T-cells do not respond to sex-hormones with changes in expression or function of telomerase [30]. Another route for transient telomerase activation involves the use of non-integrative and replication-incompetent AAVs to obtain transient expression of TERT [9, 31, 32]. This approach has been extensively studied in mice but never in humans. AAV-TERT treatment (performed by tail-vein injection) resulted in both lifespan and telomere length increase. AAV-TERT treatment also attenuated/reversed various age-associated diseases including aplastic anemia and pulmonary fibrosis, and generated beneficial effects on health and fitness (e.g., insulin resistance, osteoporosis and neuromuscular coordination) [9, 31, 32]. Taken together, these studies seem to provide a preliminary proof-of-principle that telomerase reactivation may represent an effective treatment for various aging conditions. However, it must be pointed out that all the animals employed in these investigations were of pure C57BL/6 background [9, 31, 32]. C57BL/6 mice, the most widely used inbred strain, are highly refractory to tumors. In general, AAVs can be programmed to be mostly non-integrative. However, when integration of AAV vectors into the genome does occur, even as a rare event (e.g., one cell in a million), it is associated with chromosomal deletions and rearrangements [33] and the integration occurs mainly into active genes [34] often leading to cancer [35]. Taking this into account, AAV-TERT therapy in humans (definitely not cancer-resistant) could pose high risk for the general health of the patient/individual especially the older population who may have already accumulated many premalignant alterations. In addition, exogenous TERT expression was detected at high levels for at least 8-months after AAV-TERT treatment [9, 31, 32] and steady expression of telomerase for such a time window could be too extensive to be considered safe in humans.

In summary, viral vector genomes have been modified by deleting some areas of their genomes so that their replication becomes deranged and it makes them more safe, but the system has some problems, such as their marked immunogenicity that causes induction of inflammatory system leading to degeneration of transduced tissue; toxin production causing, in turn, cell death and insertional mutagenesis [36].

Modified nucleoside-containing mRNA is believed to be non-integrating and has been recently used to transiently elevate, in vitro, levels of diverse proteins encoded by the transfected mRNA [37-39]. In particular, in vitro delivery of mRNA encoding for full-length TERT (up to three successive treatments) has been reported to transiently (24-48 hours) increase telomerase activity, lengthen telomeres and extend the replicative lifespan of normal human fibroblasts and myoblasts [40]. Importantly, delivery of TERT mRNA avoided cell immortalization and delayed expression of senescent markers [40]. This technology appears to be safer compared to viral delivery of TERT under the control of an inducible promoter and delivery of TERT using vectors based on adenovirus or adeno-associated virus. However, despite having use potential in stimulated T-cells in vitro, the delivery of hTERT mRNA may not be the ideal strategy for human interventions (especially in vivo). First, in order to be successful this strategy requires cells that can properly generate functional enzyme: once TERT is translated, it needs to undergo proper post-translational modifications, proper folding, and assembly not only with hTR but also with several other proteins such as DKC1 (Dsykerin), NOP10, TCAB1, TPP1, RTEL1, PARN and NAF1 that are essential for telomerase to bind the telomere ends and exert its full reverse transcriptase activity [41]. TERT is one of the most tightly regulated genes in the entire genome due to the strict correlation between its expression and cell growth and, in some cases, transformation. It is therefore reasonable that many cell types in the human body downregulate or silence genes encoding for “accessory” proteins important for telomerase activity.

In addition, numerous genetic diseases are caused by defects in the telomere maintenance machinery [41]. These disorders, often referred to as telomeropathies, are all characterized by one common causal molecular mechanism: the detrimental response to unprotected (critically shortened) telomeres. These diseases originate from mutations that do not necessarily involve TERT but often involve one of the several telomerase-associated proteins (DKC1, NOP10, TCAB1, TPP1, RTEL1, PARN and NAF1). In addition, patients with hTERC mutations would not make fully active telomerase with introduced TERT mRNA. Thus, delivery of TERT mRNA would not universally promote telomere lengthening in all cell types and would be potentially inefficient in treating some patients suffering from severe telomeropathy-related symptoms such as immunodeficiency, pulmonary fibrosis, cardiovascular diseases and bone marrow failure [41].

In theory, protein delivery represents the safest approach, both in vitro and in vivo, to express the activity of a gene product that for various reasons is impaired or absent. Thus, intracellular delivery of active telomerase holoenzyme (or eventually hTERT protein) represents not only a safe method but also an efficacy strategy since it circumvents most of the complicated regulatory steps and limitations associated with the other techniques discussed above. The inventors are the first to investigate this route and have shown that telomerase holoenzyme can be successfully transferred into cells to enhance telomerase function, thereby lengthening telomeres. These and other aspects of the disclosure are set out in detail below.

### I. TELOMERASE

Telomeres are protective structures that are found at the end of linear eukaryotic chromosomes consisting of multiple copies of TTAGGG DNA repeats. Telomeres are associated with six proteins; telomeric repeat binding factor (TRF)1, TRF2, TIN2, Rap1, TPP1 and POT1, which all together are called the shelterin complex [42]. Human telomeres are protected from the cellular machinery that would normally treat the end of a linear DNA strand as being broken and needing repair. The two major telomeric binding proteins, TRF1 and TRF2 are expressed in all human cells and are associated with the telomeric repeat DNA sequences throughout the cell cycle [43]. TRF1 and TRF2 are known to associate with hRap1 and the Mre11/Rad50/Nbs1 DNA repair complex [44, 45]. TRF2 is also known to bind to other DNA damage detection and repair factors, such as Ku70/80 heterodimer [46, 47]. Heterogeneous nuclear RNPs (hnRNPs), ataxia-telangiectasia mutated (ATM) kinase, and poly(ADP-ribose) polymerase (PARP) have been identified as having an effect on telomere length [48-55]. The far 3′ end comprising the telomere terminus has a single stranded overhang that can form a higher ordered structure called the t-loop [56]. These collective components and DNA structures are responsible for the protection and maintenance of the DNA ends.

Human telomerase ribonuclear protein (RNP) comprises a catalytic protein component (hTERT) and a 451 base pair RNA component, human telomerase RNA (hTR), that are both responsible for telomerase activity [57, 58]. The 3′ end of the hTR is similar to the box H/ACA family of small nucleolar RNAs (snoRNAs) and is essential for 3′ end processing, while the 5′ end contains the template used for the addition of telomeric sequences to the chromosome ends [59, 60]. The 5′ end also contains a pseudoknot that may be important for telomerase function, as well as a 6 base pair U-rich tract necessary for interaction with hnRNPs C1 and C2 [61, 62].

Several other proteins have been identified as associating with the human telomerase RNP. For example, the vault protein TEP1 was first identified, as well as the snoRNA binding proteins dyskerin and hGAR1, which bind to the 3′ end of hTR. The chaperone proteins p23/hsp90 have also been identified as binding partners and are thought to be involved in the formation of an active telomerase assembly [63]. The La autoantigen, which is involved in the assembly of other RNA particles and maturation of tRNAs, has been shown to interact with telomerase RNP and to have expression levels that correlate with telomere length [64].

Telomeres in all normal somatic cells undergo progressive shortening with each cell division due to an end replication problem, eventually resulting in cellular senescence. The end replication problem results from DNA replication being bidirectional, while DNA polymerase is unidirectional and must initiate replication from a primer. Therefore, each round of DNA replication leaves approximately 50-200 base pairs of DNA unreplicated at the 3′ end of the each DNA strand forming the chromosome. If left unchecked, the chromosome ends would become progressively shorter after each round of DNA replication. Replication-dependent telomeric shortening can be counteracted by telomerase, which adds TTAGGG repeats to the end of linear chromosomes.

Telomerase is a reverse transcriptase because of its action of copying the short RNA template sequence within the hTR into DNA. Unlike retroviral reverse transcriptases, telomerase specializes in making the short tandem repeats found at the ends of chromosomes [65]. The protein component of telomerase, hTERT, includes reverse transcriptase motifs and the core structure of the hTR component includes a pseudoknot, which is a part of the RNA that interacts strongly with the TERT protein component.

Telomerase expression is tightly regulated in normal human cells, where it is found active in stem cells and germ cells. In other normal cell types, the levels of telomerase are typically too low to sustain telomere length through the lifetime of an average human [18, 19].

### II. PROTEIN PURIFICATION AND DELIVERY

The present disclosure, in one aspect, relates to the production and formulation of telomerase holoenzymes complexes as well as their delivery to cells, tissues or subjects. In general, recombinant production of proteins is well known and is therefore not described in detail here.

A. Production and Purification of Telomerase Holoenzyme

1. Production

Detailed informations about development and overexpression of recombinant human telomerase (hTERT+hTR) and about generation of the stable cell line “Super H1299” are found in the Examples below. In addition, it should be pointed out that in some experiments, both present and future, modifications about development, production and purification of the recombinant enzyme will be employed. The following list includes possible modifications:
1) Additional cell lines for overexpression of recombinant telomerase: FDA approved cell lines for production of human recombinant proteins (e.g., HEK293, PER.C6, CHO, P. pastoris).
2) Additional TERT TAGs for purification purposes: a) 3× Flag-GS10-TERT; b) HA-GS10-TERT; c) ZZ-TEV-SS-TERT; d) Biotin-TEV-cMYC-TERT.
   All tags will have an N-terminus localization exactly as explained for our developed biotin-tag.
   In some experiments the tag will be removed after purification by protease-specific cleavage (TEV site).
3) Additional modifications to the TERT sequence: phospho-site substitutions to analyze the impact of phosphorylation events or lack thereof on recombinant telomerase activity, stability and processivity at the telomeres.

Phosphorylation (addition of a phosphate group to the lateral chain of an amino acid) is a common mechanism employed by the cell to activate or deactivate a protein as a form of regulation. Within cells, proteins are usually phosphorylated at serine, tyrosine and threonine. Some non-phosphorylated amino acids (e.g., aspartatic acid) appear chemically similar to phosphorylated amino acids (e.g., phospho-serine). Therefore, if a serine is replaced with aspartatic acid or glutamic acid in proteins whose activity, stability or processivity is enhanced by phosphorylation in that residue, as a result the protein may constitutively maintain a higher level of activity, stability or processivity. Subsequently, replacing serine, tyrosine or threonine with alanine abolishes phosphorylation at the amino acid residue.

In some embodiments recombinant telomerase has/will have four modified residues:
i) serine227 replaced by aspartatic acid,
ii) serine 824 replaced by aspartatic acid,
iii) serine 921 replaced by aspartatic acid, and/or
iv) threonine 249 replaced by alanine

2. Purification

It will be desirable to purify telomerase holoenzyme according to the present disclosure. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present disclosure concern the purification, and in particular embodiments, the substantial purification, of an encoded protein or peptide. The term “purified protein” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein composition that has been subjected to fractionation to remove various other components, and which composition substantially retains its expressed biological activity. Where the term “substantially purified” is used, this designation will refer to a composition in which the protein forms the major component of the composition, such as constituting about 50%, about 60%, about 70%, about 80%, about 90%, about 95% or more of the proteins in the composition.

Various methods for quantifying the degree of purification of the protein will be known to those of skill in the art in light of the present disclosure. These include, for example, determining the specific activity of an active fraction, or assessing the amounts of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low-pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE [66]. It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone-spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present disclosure is discussed below.

In a specific aspect, as described in greater detail on the Examples, telomerase holoenzyme was purified by the following general methods. Recombinant, telomerase-expressing cells were lysed following culture, and supernatant collected. Gradient ultra-centrifugation was performed and fractionated into 11 fractions (1 mL each). The last 5 fractions contained almost all telomerase activity. These fractions were pooled together and incubated with monomeric avidin beads, after which the beads were subjected to microbiospin chromatography. Flow-through was collected and beads were washed. Enriched telomerase was then eluted into 3 fractions, which were pooled together and subjected to bead-based chromatography. The flow-through was collected and the beads washed, after which telomerase was eluted. Elution fractions (E2, E3 and E4) were pooled together and used for subsequent assays and experiments.

B. Cell Delivery

The present disclosure contemplates the use of a cell permeability peptide (CPPs, also called a cell delivery peptide, or cell transduction domain) linked to telomerase. The intrinsic property of CPPs indicates that they may be potential components of future drugs and disease diagnostic agents [67, 68]. CPPs are relatively simple to synthesize and characterize and are able to deliver conjugated bioactive proteins inside cells, primarily via endocytosis and in a non-toxic manner. Importantly, CPPs are passive and nonselective (universally applicable to all cell types) but can also be functionalized or chemically modified to create effective delivery vectors that target specific cells or tissues (or a specific cell type in a heterogeneous cell population such as PBMC). Therefore, CPPs provide a useful platform for the possible development of medical treatments using complex proteins, such as telomerase, that had long been considered improbable for therapy.

The inventors have employed CPPs to transiently deliver purified telomerase holoenzyme to normal young and aged antigen-stimulated human peripheral blood mononuclear cells (PBMCs) and lung fibroblasts (IMR-90). In particular, the efficacy of CPPs was combined with a recently developed method reporting an active uptake mechanism in which a NaCl-mediated hyperosmolarity triggers macropinocytotic uptake and intracellular release of exogenous proteins [69] (telomerase holoenzyme is eluted in a specific NaCl/HNa₂PO4 buffer which is characterized by high osmolarity).

CPPs have been described in the art and are generally characterized as short amphipathic or cationic peptides and peptide derivatives, often containing multiple lysine and arginine residues [70]. Other examples are shown in Table 1, below.

TABLE 1
CDD/CTD PEPTIDES

| | SEQ |
|---|---|
| | ID |
| | NO: |
| GALFLGWLGAAGSTMGAKKKRKV | 1 |
| RQIKIWFQNRRMKWKK | 2 |
| RRMKWKK | 3 |
| RRWRRWWRRWWRRWRR | 4 |
| RGGRLSYSRRRFSTSTGR | 5 |
| YGRKKRRQRRR | 6 |
| RKKRRQRRR | 7 |
| YARAAARQARA | 8 |
| RRRRRRRR | 9 |
| KKKKKKKK | 10 |
| GWTLNSAGYLLGKINLKALAALAKXIL | 11 |
| LLILLRRRIRKQANAHSK | 12 |
| SRRHHCRSKAKRSRHH | 13 |
| NRARRNRRRVR | 14 |
| RQLRIAGRRLRGRSR | 15 |
| KLIKGRTPIKFGK | 16 |
| RRIPNRRPRR | 17 |
| KLALKLALKALKAALKLA | 18 |
| KLAKLAKKLAKLAK | 19 |
| GALFLGFLGAAGSTNGAWSQPKKKRKV | 20 |
| KETWWETWWTEWSQPKKKRKV | 21 |
| LKKLLKKLLKKLLKKLLKKL | 22 |
| QAATATRGRSAASRPTERPRAPARSASRPRRPVE | 23 |
| MGLGLHLLVLAAALQGAKSKRKV | 24 |
| AAVALLPAVLLALLAPAAANYKKPKL | 25 |
| MANLGYWLLALFVTMWTDVGLCKKRPKP | 26 |
| LGTYTQDFNKFHTFPQTAIGVGAP | 27 |
| DPKGDPKGVTVTVTVTVTGKGDPXPD | 28 |
| PPPPPPPPPPPPPP | 29 |
| VRLPPPVRLPPPVRLPPP | 30 |
| PRPLPPPRPG | 31 |
| SVRRRPRPPYLPRPRPPPFFPPRLPPRIPP | 32 |
| TRSSRAGLQFPVGRVHRLLRK | 33 |
| GIGKFLHSAKKFGKAFVGEIMNS | 34 |
| KWKLFKKIEKVGQNIRDGIIKAGPAVAVVGQATQIAK | 35 |
| ALWMTLLKKVLKAAAKAALNAVLVGANA | 36 |
| GIGAVLKVLTTGLPALISWIKRKRQQ | 37 |
| INLKALAALAKKIL | 38 |
| GFFALIPKIISSPLPKTLLSAVGSALGGSGGQE | 39 |
| LAKWALKQGFAKLKS | 40 |
| SMAQDIISTIGDLVKWIIQTVNXFTKK | 41 |
| LLGDFFRKSKEKIGKEFKRIVQRIKQRIKDFLANLVPRTES | 42 |
| PAWRKAFRWAWRMLKKAA | 43 |
| KLKLKLKLKLKLKLKLKL | 44 |

### IV. METHODS OF TREATING CELLS

A. Cells and Culturing

As discussed above, the present disclosure provides for increasing telomere length in cells. In general, the cells treated may be any cells, but in particular, the inventors contemplate treating engineered T cells for use in adoptive immunotherapy. However, other particular cell types of interest include bone marrow derived hematopoietic stem cells, lung epithelial cells, hepatocytes, and unfertilized eggs (prior to in vitro fertilization)

The methods will involve contacting the target cell or cell population with a purified telomerase holoenzyme, as described above. In general, it is understood that “contacting” means bringing the holoenzyme into sufficient proximity of the cell or cells such that uptake mechanisms of the cell make be activated, and the holoenzyme transferred into the cell. As such, the cells may be contacted with a unit dose of the holoenzyme preparation or may be perfused with culture media containing a specified concentration of the holoenzyme, optionally where the holoenzyme in the media is replenished to maintain a specified concentration over time. The concentration of the purified recombinant telomerase holoenzyme slightly varies across batches and it mainly depends on how many cells were used for the protein purification (in our case between 100-500 million cells). After each purification, the inventors measured the total activity of 1 μl of purified telomerase by ddTRAP, a highly quantitative assay for determining the number of telomerase molecules per cell [71]. Activity is expressed in arbitrary units, with one unit corresponding to one TS primer successfully extended by telomerase and subsequently amplified during the ddTRAP protocol. In the experiments herein described the inventors consistently delivered 5×10⁹ telomerase units per million cells.

Cells may be obtained from any source, such as a human or animal, including cells from an animal to be subsequently reinfused with treated cells, i.e., autologous cell therapy. Cells may also be cell lines or cells previously engineered with one or more heterologous constructs.

B. Formulations

Where clinical applications are contemplated, cell formulations will be prepared in a form appropriate for the intended application. Generally, this will entail preparing compositions that are essentially free of pyrogens, as well as other impurities that could be harmful to cells, humans or animals.

One will generally desire to employ appropriate salts and buffers to render enzymes stable and allow for uptake by target cells. Aqueous compositions of the present disclosure comprise an effective amount of the proteins, dissolved or dispersed in a pharmaceutically acceptable carrier or aqueous medium. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present disclosure, its use in therapeutic compositions is contemplated. Supplementary active ingredients also can be incorporated into the compositions, provided they do not inactivate the enzymes or cells.

The active compositions of the present disclosure may include classic pharmaceutical preparations. By way of illustration, solutions of the active compounds as free base or pharmacologically acceptable salts can be prepared in water suitably mixed with a surfactant, such as hydroxypropylcellulose. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. It may be desired to include isotonic agents, for example, sugars or sodium chloride.

Sterile solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

Upon formulation, solutions are preferably used in a manner compatible with the dosage formulation and in such amount as is therapeutically effective (see for example, “Remington's Pharmaceutical Sciences” 15th Edition, pages 1035-1038 and 1570-1580). Some variation in dosage may occur depending on the particular target cell. The person responsible for administration will, in any event, determine the appropriate dose for the individual subject. Moreover, for human administration, preparations should meet sterility, pyrogenicity, general safety and purity standards as required by FDA Office of Biologics standards.

### IV. EXAMPLES

The following examples are included to further illustrate various aspects of the disclosure. It should be appreciated by those of skill in the art that the techniques disclosed in the examples which follow represent techniques and/or compositions discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Methods

Development and Overexpression of Recombinant Human Telomerase (hTERT+hTR) and Generation of the Stable Cell Line Super H1299. The engineered recombinant hTERT contains an in vivo biotinylation sequence, a Tev-protease cutting site, a cMyc tag before the hTERT N-terminus, adding 99 amino acid residues before the hTERT sequence. The added sequence is: MAGKAGEGEIPAPLAGTVSKILVKEGDTVKAGQTVLVLEAMKMETEINAPTDGKVE KVLVKERDAVQGGQGLIKIGVENLYFQSTMEQKLISEEDLEFT (SEQ ID NO: 45). The conserved biotinylated sequence is biotinylated at the conserved MKM site in mammalian cells. The modified hTERT plasmid and the exogenous hTR plasmid were packaged in retroviral and lentiviral vectors respectively and used to transfect and generate a stable cell line, which the inventors called Super H1299. After hygromycin selection the cells were grown and harvested on a weekly basis and used for various experiments.

Biotin tagged hTERT carried in pBabe-hygro retroviral vector was transfected into the transient packaging line PhoenixE. The virus-containing supernatant was then used to infect the stable amphotropic packaging line PA317. The PA317 cells were then selected with hygromycin and produced stable viruses that were used to infect the expressing cell line H1299. The infected H1299 cells were selected with hygromycin.

For hTR, pSSI 7661 lentiviral vector together with two helper plasmids, psPAX2 and pMD2G, were used to transfect 293 packaging cells. The virus supernatant was used to infect H1299 cells that expressed the biotinylation sequence-tagged hTERT. The infected H1299 cells were further selected with blasticidin and hygromycin.

Purification of Recombinant Telomerase from Super H1299 (3-Step). 200 million frozen cell pellets of super H1299 cells were lysed in a 1.5% CHAPS lysis buffer (10% glycerol, 1 mM EGTA pH8.0, 0.1 mM MgCl₂, 10 mM Tris-HCL, 0.01 mM PMSF, 1 unit of RiboLock RNAse inhibitor and 1 unit of PI cocktail) for 30 min rotating end over end at 4° C. Cells were then centrifuged at 17,500×g for 1 hr at 4° C. Supernatants were collected and placed in clean tubes. A 10 ml continuous glycerol gradient (10-30%) was generated with a gradient maker (glycerol, 20 mM HEPES pH7.5, 300 mM KCl, 0.1 mM MgCl₂, 0.1% Triton X-100 and 1 mM EGTA). The cell lysate sample was loaded onto the top of the gradient before ultra-centrifugation at 126,000×g for 19 hrs at 4° C. (SW41 Beckman rotor). The gradient was fractionated into 11 fractions (1 mL each). The bottom 5 fractions contained almost all telomerase activity. These 5 fractions (7-11) were pooled together and incubated with monomeric avidin beads (Peirce) for 2 hrs at 4° C. After incubation, the beads were placed into a microbiospin chromatography column (BioRad). The flow-through solution was collected and beads were washed 2 times with 5 ml buffer containing 150 mM sodium phosphate, pH 7.0 and 100 mM NaCl. The enriched telomerase was then eluted with 400 mM NaCl, 150 mM sodium phosphate buffer pH7.0 and 4 mM D-biotin (Pierce). The telomerase activity was eluted into 3 fractions of 1 ml each. These elution fractions were pooled together and incubated with the final column, SP (sulphopropyl) Sepharose Fast Flow (SPFF). SPFF resin was equilibrated in 50 mM sodium phosphate (pH 7.0) and 50 mM NaCl prior to incubation with telomerase. Telomerase was incubated with SPFF beads for 2 hrs at 4° C. After incubation, the beads were loaded into a microbiospin column. The flow-through was collected and the SPFF beads were washed 2 times with 5 ml buffer made of 20 mM sodium phosphate pH7.0 and 50 mM NaCl. Telomerase was then eluted in a NaCl salt gradient (200 mM to 500 mM in 6 steps). This was done in 6 separate elution fractions (500 μl). Eluates from 500 mM contained most of the telomerase activity. These elution fractions (E2, E3 and E4) were pooled together and used for subsequent assays and experiments.

PBMC isolation, stimulation and treatment with telomerase holoenzyme. Peripheral blood mononuclear cells (PBMCs) were isolated from peripheral blood of healthy volunteers by centrifugation with Ficoll-Paque Plus (GE Healthcare) and were then cryopreserved at −140° C. pending analysis. Cells were thawed 24 hours prior to mitogen stimulation and cultured in RPMI+GlutaMAX-I with 10% fetal bovine serum, 10 ng/ml IL-2, 1% penicillin, streptomycin and amphotericin B. After 24 h the cell suspensions were transferred into a new flask to remove the monocytes (that adhered to the flask's plastic).

PBMCs were stimulated by adding Dynabeads Human T-Activator CD3/CD28 (Life Technologies) in a 1:1 ratio. After 72 hours of stimulation Dynabeads were removed using a magnet and cells were cultured up to 35 days after stimulation. Cells were re-stimulated every 8-10 days. The percentage of live cells was determined every day by trypan blue exclusion using a TC20 Automated Cell Counter (Bio-Rad). The cell density was adjusted daily and when it exceeded 1.5×10⁶/ml, cells were diluted with fresh complete RPMI medium to a density of 1.0×10⁶/ml.

Telomerase holoenzyme was delivered three consecutive times at day 3, 6 and 9 after stimulation. Before delivery cells were centrifuged at 500 g for 15 min and resuspended in serum-free RPMI+GlutaMAX-I supplemented with 10 ng/ml IL-2 and 200 U/ml recombinant ribonuclease inhibitor. Telomerase holoenzyme in 500 mM NaCl and 50 mM sodium phosphate pH7.0 was mixed with cell penetrating peptides (Xfect kit, Protein transfection protocol, Takara) and added to the cells resuspended in serum-free media. After 1-hour incubation at 37° C. cells were centrifuged at 500 g for 15 min, resuspended in complete media (RPMI+GlutaMAX-I with 10% fetal bovine serum, 10 ng/ml IL-2, 1% penicillin, streptomycin and amphotericin B) and cultured at 37° C., 5% CO₂.

### Example 2—Results

Holoenzyme production. The inventors have successfully engineered a biotin-tagged recombinant hTERT and overexpressed it along with hTR (the functional RNA component of telomerase) in human cells. They also developed a 3-step purification procedure strategy to obtain the recombinant enzyme.

The multi-step purification procedure allowed us to obtain highly enriched, catalytically active enzyme. Importantly, the employed biotin-tag (developed by us) allowed pulling down not only telomerase but the whole reconstituted holoenzyme complex containing other essential telomerase-associated proteins such as dyskerin (DKC1) and the ribonucleoprotein NOP10 and NHP2.

PBMCs. PBMCs are a heterogeneous cell population mainly consisting of T-cells, a major component of human immune responses. T-cells remain in a resting or quiescent state when unstimulated, showing little or no proliferation activity. In contrast, upon antigen-specific activation T-cells rapidly divide and exhibit dramatic changes in gene expression [72].

Activated T-cells initiate immune responses such as discriminating between healthy and abnormal (e.g., infected or cancerous) cells in the body and are finding increased use as a prime tool for adoptive immunotherapy to treat various forms of cancer and infectious diseases such as AIDS [16, 17]. The inventors stimulated PBMC exactly as engineered CAR-T cells are activated and expanded [73] with the difference that they have not employed a WAVE bioreactor for cell culture and PBMCs were not previously transfected with the 4-1BB receptor.

ddTRAP. In order to measure telomerase activity, the inventors employed a Droplet Digital PCR assay (the ddTRAP) previously developed in their lab [71]. ddTRAP is a digital, high-throughput and highly sensitive assay that provides an absolute quantification of telomerase activity at the single cell level. Importantly, this improved technology is able to discriminate between samples having as little as 10% differences in telomerase activity, as opposed to the gel-based TRAP (still largely employed in the field but only semi-quantitative).

Telomere Shortest Length Assay (TeSLA). Telomere length was measured by using a new highly sensitive and precise assay (TeSLA, Telomere Shortest Length Assay) that was recently developed in the inventors' lab [74]. TeSLA allows to simultaneously measure both the average telomere length and the length of the shortest 20% telomeres. Importantly, as opposed to both TRF and Q-FISH (currently the gold-standard in the field) TeSLA is able to detect small variations in telomere length such as the physiological telomere attrition that occur in human immune cells over a 1-year period [74]. With TeSLA, the inventors were able to document progressive telomere shortening over a 10-day period in stimulated PBMC expanded in vitro.

The inventors successfully delivered purified telomerase holoenzyme in the cytoplasmic compartment of different normal human cell types, including resting and stimulated PBMC, and demonstrated by using ddTRAP that the delivered complex maintained a strong activity.

Next, the inventors demonstrated that delivered telomerase was subsequently trafficked to the nucleus. To this aim, they fractionated the cellular cytoplasmic and nuclear compartment and performed ddTRAP on the two separate fractions. Telomerase activity from both the cytoplasmic and the nuclear fraction was significantly increased after delivery indicating that the purified telomerase complex is able to cross the nuclear membrane (potentially through the nuclear pores) and access the nucleus.

To investigate whether delivered telomerase also maintained its ability to add TTAGGG repeats to the telomere ends and to investigate if the employed biotin-tag affected, in the cell, the enzyme ability to bind the telomeres, they measured telomere length in stimulated PBMC treated with telomerase.

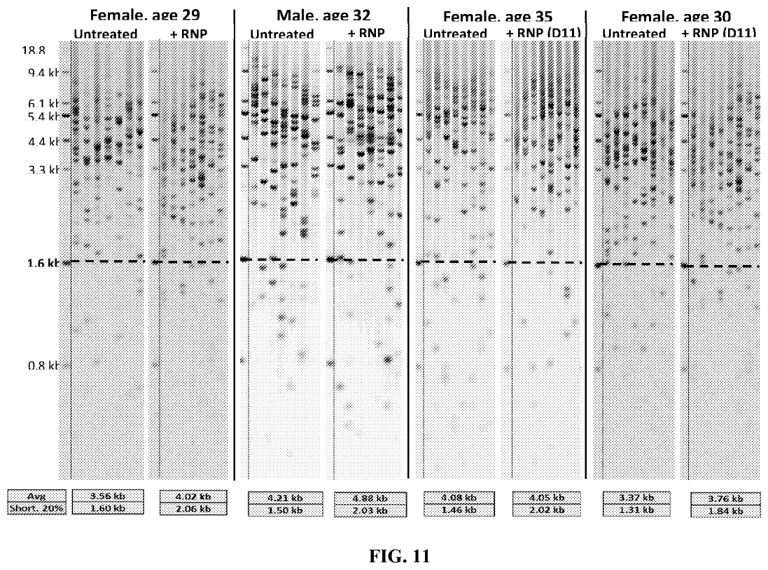

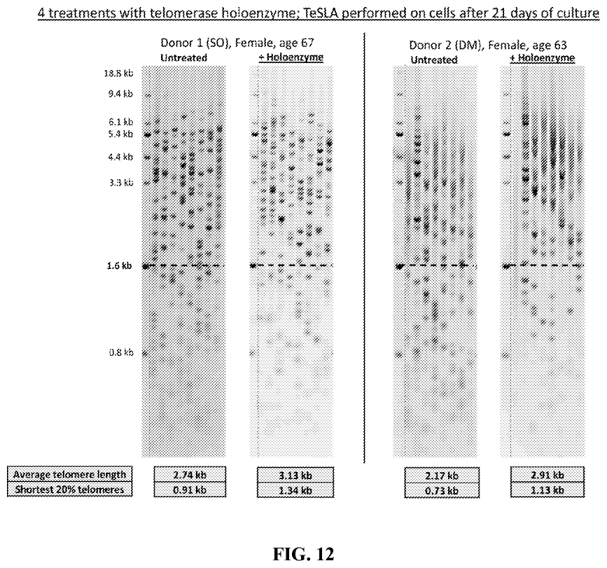

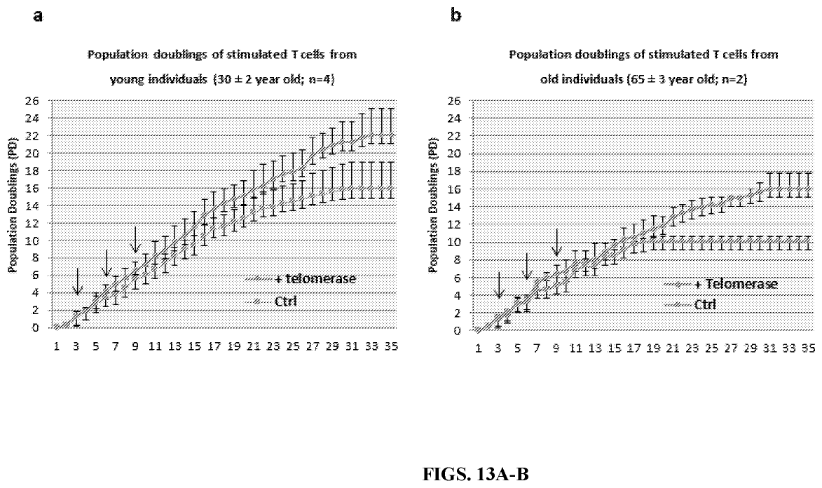

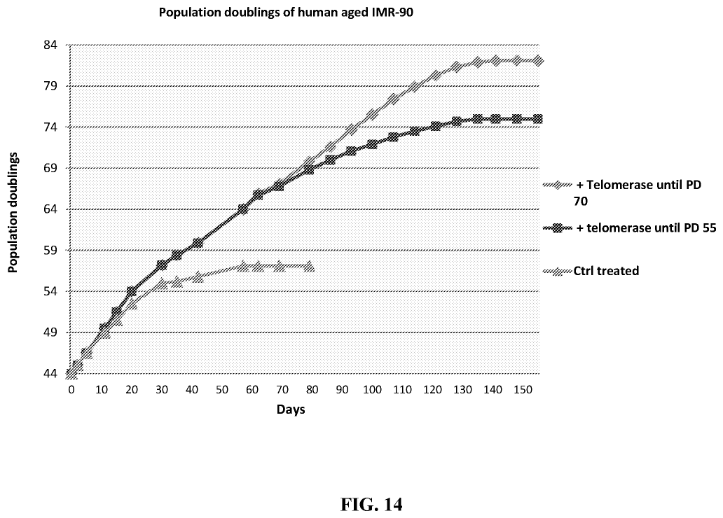

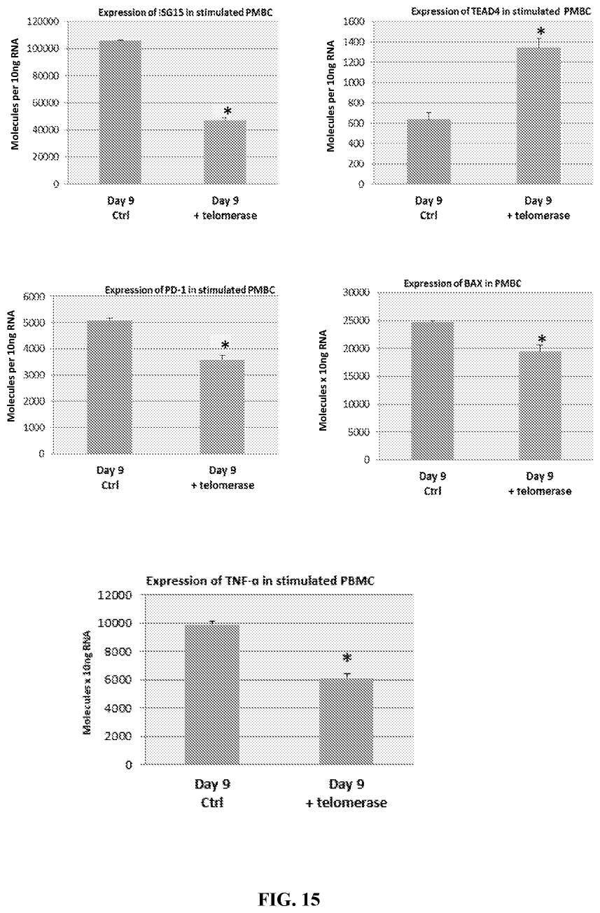

The inventors delivered telomerase holoenzyme three times (day 3, 6, and 9) to stimulated PBMC from four young adults (mean age 32±2 year-old) and two older volunteers (mean age 65±3 year-old). Telomerase delivery significantly decreased the rate of telomere shortening during rapid cell expansion (see FIG. 11 representing four individual's TeSLA profiles). Importantly, this treatment preferentially extended the length of the shortest telomeres which are believed to best correlate not only with cell viability and chromosome stability but also with various age-related diseases and phenotypes of aging [75].

The inventors next demonstrated that their treatment also extended the T-cell replicative lifespan. Cells were electronically counted every day including trypan blue exclusion until they showed no signs of growth for at least three consecutive days. They also treated aged human lung fibroblasts (every three days) and demonstrated that telomerase holoenzyme delivery can also be applied to normal telomerase negative cells and adherent cell cultures in general.

The inventors previously identified a group of genes whose expressions were directly regulated by telomere length (telomere position effects over long distances, TPE-OLD) [76, 77]. In these studies, the presence of long telomeres resulted in a telomere “chromosome loop” approaching genes up to 10 Mb away of the telomere end. In cells with short telomeres these interstitial telomere loops are lost and the same loci became separated [77]. Telomere looping promotes epigenetic regulation of gene expression (it generally silences gene expression). TPE-OLD is therefore a mechanism by which progressive telomere shortening directly leads to changes in gene expression that, in turn, could contribute to aging and disease initiation/progression long before telomeres become short enough to cause critical DNA damage responses and senescence [77].

The inventors measured by ddPCR the expression level of some of those genes and observed that the telomere lengthening induced by telomerase holoenzyme delivery was also correlated with changes in gene expression. Those genes involved in inflammatory pathways and apoptotic signaling are regulated by telomere looping and their expression level changes potentially precede cellular replicative senescence. This suggests that by preventing telomere shortening, a single hallmark of aging, this is sufficient to also alter gene expression toward a more “youthful” profile. These genes and their expression are potential biomarkers of efficacy of telomerase delivery.

The inventors analyzed whole genome expression profiles of stimulated PBMC treated with telomerase holoenzyme to compare to the untreated controls. By comparing this new data set with the one they obtained from the study of healthy vs frail centenarians, they observed that cells treated with telomerase holoenzyme specifically regulated the expression of genes that are strongly associated with healthy aging and longevity (data not shown).

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods, and in the steps or in the sequence of steps of the methods described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
[1] Blackburn E H, Collins K (2011) Telomerase: An RNP Enzyme Synthesizes DNA. Cold Spring Harbor Perspectives in Biology 3.
[2] Olovnikov A M (1973) A theory of marginotomy. The incomplete copying of template margin in enzymic synthesis of polynucleotides and biological significance of the phenomenon. J Theor Biol 41, 181-190.
[3] Shay J W (2016) Role of Telomeres and Telomerase in Aging and Cancer. Cancer Discovery 6, 584-593.
[4] Aubert G, Baerlocher G M, Vulto I, Poon S S, Lansdorp P M (2012) Collapse of Telomere Homeostasis in Hematopoietic Cells Caused by Heterozygous Mutations in Telomerase Genes. Plos Genetics 8.
[5] Epel E S, Merkin S S, Cawthon R, Blackburn E H, Adler N E, Pletcher M J, Seeman T E (2009) The rate of leukocyte telomere shortening predicts mortality from cardiovascular disease in elderly men. Aging (Albany N.Y.) 1, 81-88.
[6] Honig L S, Schupf N, Lee J H, Tang M X, Mayeux R (2006) Shorter telomeres are associated with mortality in those with APOE epsilon4 and dementia. Ann Neurol 60, 181-187.
[7] Tedone E, Arosio B, Colombo F, Ferri E, Asselineau D, Piette F, Gussago C, Belmin J, Panel S, Benlhassan K, Casati M, Bornand A, Rossi P D, Mazzola P, Annoni G, Doulazmi M, Mariani J, Porretti L, Bray D H, Mari D (2015) Leukocyte Telomere Length in Alzheimer's Disease Patients with a Different Rate of Progression. Journal of Alzheimers Disease 46, 761-769.
[8] Tomas-Loba A, Flores I, Fernandez-Marcos P J, Cayuela M L, Maraver A, Tejera A, Borras C, Matheu A, Klatt P, Flores J M, Vina J, Serrano M, Blasco M A (2008) Telomerase reverse transcriptase delays aging in cancer-resistant mice. Cell 135, 609-622.
[9] Bernardes de Jesus B, Vera E, Schneeberger K, Tejera A M, Ayuso E, Bosch F, Blasco M A (2012) Telomerase gene therapy in adult and old mice delays aging and increases longevity without increasing cancer. EMBO Mol Med 4, 691-704.
[10] Hiyama E, Hiyama K (2007) Telomere and telomerase in stem cells. Br J Cancer 96, 1020-1024.
[11] Huang E E, Tedone E, O'Hara R, Cornelius C, Lai T P, Ludlow A, Wright W E, Shay J W (2017) The Maintenance of Telomere Length in CD28+ T Cells During T Lymphocyte Stimulation. Sci Rep 7, 6785.
[12] Effros R B, Dagarag M, Spaulding C, Man J (2005) The role of CD8+ T-cell replicative senescence in human aging. Immunol Rev 205, 147-157.
[13] Labarriere N, Pandolfino M C, Gervois N, Khammari A, Tessier M H, Dreno B, Jotereau F (2002) Therapeutic efficacy of melanoma-reactive TIL injected in stage III melanoma patients. Cancer Immunol Immunother 51, 532-538.
[14] Zhou J, Shen X, Huang J, Hodes R J, Rosenberg S A, Robbins P F (2005) Telomere length of transferred lymphocytes correlates with in vivo persistence and tumor regression in melanoma patients receiving cell transfer therapy. J Immunol 175, 7046-7052.
[15] Shen X, Zhou J, Hathcock K S, Robbins P, Powell D J, Jr., Rosenberg S A, Hodes R J (2007) Persistence of tumor infiltrating lymphocytes in adoptive immunotherapy correlates with telomere length. J Immunother 30, 123-129.
[16] Muranski P, Restifo N P (2009) Adoptive immunotherapy of cancer using CD4(+) T cells. Current Opinion in Immunology 21, 200-208.
[17] Johnson L A, Heemskerk B, Powell D J, Cohen C J, Morgan R A, Dudley M E, Robbins P F, Rosenberg S A (2006) Gene transfer of tumor-reactive TCR confers both high avidity and tumor reactivity to nonreactive peripheral blood mononuclear cells and tumor-infiltrating lymphocytes. Journal of Immunology 177, 6548-6559.
[18] Morales C P, Holt S E, Ouellette M, Kaur K J, Yan Y, Wilson K S, White M A, Wright W E, Shay J W (1999) Absence of cancer-associated changes in human fibroblasts immortalized with telomerase. Nat Genet 21, 115-118.
[19] Rufer N, Migliaccio M, Antonchuk J, Humphries R K, Roosnek E, Lansdorp P M (2001) Transfer of the human telomerase reverse transcriptase (TERT) gene into T lymphocytes results in extension of replicative potential. Blood 98, 597-603.
[20] Anson D S (2004) The use of retroviral vectors for gene therapy-what are the risks? A review of retroviral pathogenesis and its relevance to retroviral vector-mediated gene delivery. Genet Vaccines Ther 2, 9.
[21] Fischer A, Hacein-Bey-Abina S, Lagresle C, Garrigue A, Cavazana-Calvo M (2005) [Gene therapy of severe combined immunodeficiency disease: proof of principle of efficiency and safety issues. Gene therapy, primary immunodeficiencies, retrovirus, lentivirus, genome]. Bull Acad Natl Med 189, 779-785; discussion 786-778.
[22] Shay J W, Reddel R R, Wright W E (2012) Cancer. Cancer and telomeres—an ALTernative to telomerase. Science 336, 1388-1390.
[23] Calado R T, Yewdell W T, Wilkerson K L, Regal J A, Kajigaya S, Stratakis C A, Young N S (2009) Sex hormones, acting on the TERT gene, increase telomerase activity in human primary hematopoietic cells. Blood 114, 2236-2243.
[24] Fauce S R, Jamieson B D, Chin A C, Mitsuyasu R T, Parish S T, Ng H L, Kitchen C M, Yang 00, Harley C B, Effros R B (2008) Telomerase-based pharmacologic enhancement of antiviral function of human CD8+T lymphocytes. J Immunol 181, 7400-7406.
[25] Molgora B, Bateman R, Sweeney G, Finger D, Dimler T, Effros R B, Valenzuela H F (2013) Functional assessment of pharmacological telomerase activators in human T cells. Cells 2, 57-66.
[26] Ni M, Chen Y, Fei T, Li D, Lim E, Liu X S, Brown M (2013) Amplitude modulation of androgen signaling by c-MYC. Genes Dev 27, 734-748.
[27] Salvador L, Singaravelu G, Harley C B, Flom P, Suram A, Raffaele J M (2016) A Natural Product Telomerase Activator Lengthens Telomeres in Humans: A Randomized, Double Blind, and Placebo Controlled Study. Rejuvenation Res 19, 478-484.
[28] Townsley D M, Dumitriu B, Liu D, Biancotto A, Weinstein B, Chen C, Hardy N, Mihalek A D, Lingala S, Kim Y J, Yao J, Jones E, Gochuico B R, Heller T, Wu C O, Calado R T, Scheinberg P, Young N S (2016) Danazol Treatment for Telomere Diseases. N Engl J Med 374, 1922-1931.
[29] Vasko T, Kaifie A, Stope M B, Kraus T, Ziegler P (2017) Telomeres and Telomerase in Hematopoietic Dysfunction: Prognostic Implications and Pharmacological Interventions. Int J Mol Sci 18.
[30] Benko A L, Olsen N J, Kovacs W J (2012) Estrogen and telomerase in human peripheral blood mononuclear cells. Mol Cell Endocrinol 364, 83-88.
[31] Bar C, Povedano J M, Serrano R, Benitez-Buelga C, Popkes M, Formentini I, Bobadilla M, Bosch F, Blasco M A (2016) Telomerase gene therapy rescues telomere length, bone marrow aplasia, and survival in mice with aplastic anemia. Blood 127, 1770-1779.
[32] Povedano J M, Martinez P, Serrano R, Tejera A, Gomez-Lopez G, Bobadilla M, Flores J M, Bosch F, Blasco M A (2018) Therapeutic effects of telomerase in mice with pulmonary fibrosis induced by damage to the lungs and short telomeres. Elife 7.
[33] Miller D G, Rutledge E A, Russell D W (2002) Chromosomal effects of adeno-associated virus vector integration. Nat Genet 30, 147-148.
[34] Nakai H, Montini E, Fuess S, Storm T A, Grompe M, Kay M A (2003) AAV serotype 2 vectors preferentially integrate into active genes in mice. Nat Genet 34, 297-302.
[35] Donsante A, Miller D G, Li Y, Vogler C, Brunt E M, Russell D W, Sands M S (2007) AAV vector integration sites in mouse hepatocellular carcinoma. Science 317, 477.
[36] Gardlik R, Palffy R, Hodosy J, Lukacs J, Turna J, Celec P (2005) Vectors and delivery systems in gene therapy. Med Sci Monit 11, RA110-121.
[37] Kariko K, Muramatsu H, Keller J M, Weissman D (2012) Increased erythropoiesis in mice injected with submicrogram quantities of pseudouridine-containing mRNA encoding erythropoietin. Mol Ther 20, 948-953.
[38] Kormann M S, Hasenpusch G, Aneja M K, Nica G, Flemmer A W, Herber-Jonat S, Huppmann M, Mays L E, Illenyi M, Schams A, Griese M, Bittmann I, Handgretinger R, Hartl D, Rosenecker J, Rudolph C (2011) Expression of therapeutic proteins after delivery of chemically modified mRNA in mice. Nat Biotechnol 29, 154-157.
[39] Wang Y, Su H H, Yang Y, Hu Y, Zhang L, Blancafort P, Huang L (2013) Systemic delivery of modified mRNA encoding herpes simplex virus 1 thymidine kinase for targeted cancer gene therapy. Mol Ther 21, 358-367.
[40] Ramunas J, Yakubov E, Brady J J, Corbel S Y, Holbrook C, Brandt M, Stein J, Santiago J G, Cooke J P, Blau H M (2015) Transient delivery of modified mRNA encoding TERT rapidly extends telomeres in human cells. FASEB J 29, 1930-1939.
[41] Holohan B, Wright W E, Shay J W (2014) Cell biology of disease: Telomeropathies: an emerging spectrum disorder. J Cell Biol 205, 289-299.
[42] de Lange T (2005) Shelterin: the protein complex that shapes and safeguards human telomeres. Genes Dev 19, 2100-2110.
[43] Shay J W, Zou Y, Hiyama E, Wright W E (2001) Telomerase and cancer. Hum Mol Genet 10, 677-685.
[44] Li B, Oestreich S, de Lange T (2000) Identification of human Rap1: implications for telomere evolution. Cell 101, 471-483.
[45] Zhu X D, Kuster B, Mann M, Petrini J H, de Lange T (2000) Cell-cycle-regulated association of RAD50/MRE11/NBS1 with TRF2 and human telomeres. Nat Genet 25, 347-352.
[46] Bianchi A, de Lange T (1999) Ku binds telomeric DNA in vitro. J Biol Chem 274, 21223-21227.
[47] Hsu H L, Gilley D, Galande S A, Hande M P, Allen B, Kim S H, Li G C, Campisi J, Kohwi-Shigematsu T, Chen D J (2000) Ku acts in a unique way at the mammalian telomere to prevent end joining. Genes Dev 14, 2807-2812.
[48] McKay S J, Cooke H (1992) hnRNP A2/B1 binds specifically to single stranded vertebrate telomeric repeat TTAGGGn. Nucleic Acids Res 20, 6461-6464.
[49] LaBranche H, Dupuis S, Ben-David Y, Bani M R, Wellinger R J, Chabot B (1998) Telomere elongation by hnRNP A1 and a derivative that interacts with telomeric repeats and telomerase. Nat Genet 19, 199-202.
[50] Eversole A, Maizels N (2000) In vitro properties of the conserved mammalian protein hnRNP D suggest a role in telomere maintenance. Mol Cell Biol 20, 5425-5432.
[51] Dallaire F, Dupuis S, Fiset S, Chabot B (2000) Heterogeneous nuclear ribonucleoprotein A1 and UP1 protect mammalian telomeric repeats and modulate telomere replication in vitro. J Biol Chem 275, 14509-14516.
[52] Smilenov L B, Morgan S E, Mellado W, Sawant S G, Kastan M B, Pandita T K (1997) Influence of ATM function on telomere metabolism. Oncogene 15, 2659-2665.
[53] Smilenov L B, Dhar S, Pandita T K (1999) Altered telomere nuclear matrix interactions and nucleosomal periodicity in ataxia telangiectasia cells before and after ionizing radiation treatment. Mol Cell Biol 19, 6963-6971.
[54] Wood L D, Halvorsen T L, Dhar S, Baur J A, Pandita R K, Wright W E, Hande M P, Calaf G, Hei T K, Levine F, Shay J W, Wang J J, Pandita T K (2001) Characterization of ataxia telangiectasia fibroblasts with extended life-span through telomerase expression. Oncogene 20, 278-288.
[55] d'Adda di Fagagna F, Hande M P, Tong W M, Lansdorp P M, Wang Z Q, Jackson S P (1999) Functions of poly(ADP-ribose) polymerase in controlling telomere length and chromosomal stability. Nat Genet 23, 76-80.
[56] Griffith J D, Comeau L, Rosenfield S, Stansel R M, Bianchi A, Moss H, de Lange T (1999) Mammalian telomeres end in a large duplex loop. Cell 97, 503-514.
[57] Bodnar A G, Ouellette M, Frolkis M, Holt S E, Chiu C P, Morin G B, Harley C B, Shay J W, Lichtsteiner S, Wright W E (1998) Extension of life-span by introduction of telomerase into normal human cells. Science 279, 349-352.
[58] Weinrich S L, Pruzan R, Ma L, Ouellette M, Tesmer V M, Holt S E, Bodnar A G, Lichtsteiner S, Kim N W, Trager J B, Taylor R D, Carlos R, Andrews W H, Wright W E, Shay J W, Harley C B, Morin G B (1997) Reconstitution of human telomerase with the template RNA component hTR and the catalytic protein subunit hTRT. Nat Genet 17, 498-502.
[59] Narayanan A, Lukowiak A, Jady B E, Dragon F, Kiss T, Terns R M, Terns M P (1999) Nucleolar localization signals of box H/ACA small nucleolar RNAs. EMBO J 18, 5120-5130.
[60] Greider C W, Blackburn E H (1987) The telomere terminal transferase of Tetrahymena is a ribonucleoprotein enzyme with two kinds of primer specificity. Cell 51, 887-898.
[61] Gilley D, Blackburn E H (1999) The telomerase RNA pseudoknot is critical for the stable assembly of a catalytically active ribonucleoprotein. Proc Natl Acad Sci USA 96, 6621-6625.
[62] Ford L P, Suh J M, Wright W E, Shay J W (2000) Heterogeneous nuclear ribonucleoproteins C1 and C2 associate with the RNA component of human telomerase. Mol Cell Biol 20, 9084-9091.
[63] Holt S E, Aisner D L, Baur J, Tesmer V M, Dy M, Ouellette M, Trager J B, Morin G B, Toft D O, Shay J W, Wright W E, White M A (1999) Functional requirement of p23 and Hsp90 in telomerase complexes. Genes Dev 13, 817-826.
[64] Ford L P, Shay J W, Wright W E (2001) The La antigen associates with the human telomerase ribonucleoprotein and influences telomere length in vivo. RNA 7, 1068-1075.
[65] Blackburn E H (2005) Telomerase and Cancer: Kirk A. Landon—AACR prize for basic cancer research lecture. Mol Cancer Res 3, 477-482.
[66] Capaldi R A, Bell R L, Branchek T (1977) Changes in order of migration of polypeptides in complex III and cytochrome C oxidase under different conditions of SDS polyacrylamide gel electrophoresis. Biochem Biophys Res Commun 74, 425-433.
[67] Copolovici D M, Langel K, Eriste E, Langel U (2014) Cell-penetrating peptides: design, synthesis, and applications. ACS Nano 8, 1972-1994.
[68] Dinca A, Chien W M, Chin M T (2016) Intracellular Delivery of Proteins with Cell-Penetrating Peptides for Therapeutic Uses in Human Disease. Int J Mol Sci 17, 263.
[69] D'Astolfo D S, Pagliero R J, Pras A, Karthaus W R, Clevers H, Prasad V, Lebbink R J, Rehmann H, Geijsen N (2015) Efficient intracellular delivery of native proteins. Cell 161, 674-690.
[70] Fischer P M (2007) Cellular uptake mechanisms and potential therapeutic utility of peptidic cell delivery vectors: progress 2001-2006. Med Res Rev 27, 755-795.
[71] Ludlow A T, Robin J D, Sayed M, Litterst C M, Shelton D N, Shay J W, Wright W E (2014) Quantitative telomerase enzyme activity determination using droplet digital PCR with single cell resolution. Nucleic Acids Res 42, e104.
[72] Zhao S R, Fung-Leung W P, Bittner A, Ngo K, Liu X J (2014) Comparison of RNA-Seq and Microarray in Transcriptome Profiling of Activated T Cells. Plos One 9.
[73] Hollyman D, Stefanski J, Przybylowski M, Bartido S, Borquez-Ojeda O, Taylor C, Yeh R, Capacio V, Olszewska M, Hosey J, Sadelain M, Brentj ens RJ, Riviere I (2009) Manufacturing validation of biologically functional T cells targeted to CD19 antigen for autologous adoptive cell therapy. J Immunother 32, 169-180.
[74] Lai T P (2017) A method for measuring the distribution of the shortest telomeres in cells and tissues. 8, 1356.
[75] Hemann M T, Strong M A, Hao L Y, Greider C W (2001) The shortest telomere, not average telomere length, is critical for cell viability and chromosome stability. Cell 107, 67-77.
[76] Lou Z, Wei J, Riethman H, Baur J A, Voglauer R, Shay J W, Wright W E (2009) Telomere length regulates ISG15 expression in human cells. Aging (Albany N.Y.) 1, 608-621.
[77] Robin J D, Ludlow A T, Batten K, Magdinier F, Stadler G, Wagner K R, Shay J W, Wright W E (2014) Telomere position effect: regulation of gene expression with progressive telomere shortening over long distances. Genes Dev 28, 2464-2476.

1. A method of increasing telomere length and/or increasing the proliferative capacity of a cell comprising:
(i) providing a population of cells;
(ii) contacting at least a first portion said population of cells with a purified recombinant telomerase holoenzyme; and
(iii) measuring the expression of one or more target genes regulated by telomere length in a cell from said first portion.

2. The method of claim 1, further comprising:
(iv) introducing a second cell from said first portion into a subject when one or more of said target genes shows an expression profile indicative of telomerase activity as compared to an untreated cell, such as an untreated cell from a second portion of said population of cells.

3. The method of claim 1, further comprising measuring the expression of one or more target genes regulated by telomere length in a third cell of said population of cells prior to step (ii).

4. The method of claim 1, wherein said one or more target genes is/are ISG15, TEAD4, PD-1, and/or BAX.

5. The method of claim 1, wherein said population of cells are PBMCs.

6. The method of claim 1, wherein said population of cells are T cells, such as a CD3⁺/CD28⁺ T cell.

7. The method of claim 1, further comprising removing said population of cells from a subject prior to step (i).

8. The method of claim 2, wherein said subject is a human subject.

9. The method of claim 2, wherein said subject is a humanized mouse, such as a NOD SCID gamma mouse with umbilical cord blood stem cells.

10. The method of claim 1, wherein said telomerase holoenzyme is coupled to a cell permeability peptide.

11. A method of increasing a cell's proliferative capacity comprising:
(i) providing a population of cells;
(ii) contacting said a first portion of said population of cells with a recombinant telomerase holoenzyme;
(iii) measuring the total number of cell divisions that a first cell from said first portion performs before senescence or apoptosis are triggered;
(iv) measuring the total number of cell divisions that a cell from a second but non-telomerase treated portion of said population of cells performs before senescence or apoptosis are triggered; and
(v) determining whether a second cell from said first portion does not exhibit a characteristic of cancer.

12. The method of claim 11, further comprising:
(iv) introducing a third cell from said first portion into a subject when the total number of cell divisions measured in step (iii) is greater than in step (iv), and when said second cell from said first portion does not exhibit a characteristic of cancer.

13. The method of claim 11, further comprising measuring telomere length and/or the expression of one or more target genes regulated by telomere length (a) as part of step (iii) or (b) if a fourth cell from said population of cells prior to step (ii).

14. The method of claim 13, wherein said one or more target genes is/are ISG15, TEAD4, PD-1, and/or BAX.

15. The method of claim 11, wherein said cell first population of cells are PBMCs.

16. The method of claim 11, wherein said first population of cells are T cells, such as a CD3⁺/CD28⁺ T cells.

17. The method of claim 11, further comprising removing said population of cells from said subject prior to step (i).

18. The method of claim 12, wherein said subject is a human subject.

19. The method of claim 12, wherein said subject is a humanized mouse, such as a NOD SCID gamma mouse with umbilical cord blood stem cells.

20. The method of claim 11, wherein said telomerase holoenzyme is coupled to a cell permeability peptide.
